(19) United States
(12) Patent Application Publication
(10) Pub. No.: US 2013/0231727 A1
(43) Pub. Date: Sep. 5, 2013

(54) LEAD WITH BIOABSORBABLE METALLIC FIXATION STRUCTURE

(71) Applicants: Mark Carlson, Calabasas, CA (US); Gene A. Bornzin, Simi Valley, CA (US)

(72) Inventors: Mark Carlson, Calabasas, CA (US); Gene A. Bornzin, Simi Valley, CA (US)

(73) Assignee: PACESETTER, INC., Sylmar, CA (US)

(21) Appl. No.: 13/412,417

(22) Filed: Mar. 5, 2012

(51) Int. Cl. A61N 1/05

(52) U.S. Cl. 607/126

(57) ABSTRACT

In one embodiment, an implantable medical lead includes a lead connector end, a tubular body, at least one electrode and at least one fixation structure. The lead connector end is configured to couple to the implantable pulse generator. The tubular body extends distally from the lead connector end and includes a distal portion distally terminating in a distal end. The at least one electrode is located on the distal portion. The at least one fixation structure is located on the distal portion and includes a bioabsorbable metal. For example, the bioabsorbable metal may be iron, an iron alloy with 35% manganese, or a magnesium alloy. The bioabsorbable metal is configured such that the at least one fixation structure will last long enough at an implantation site so as to secure the distal portion of the tubular body in place via fibrotic tissue.

### FIELD OF THE INVENTION

Aspects of the present invention relate to medical apparatus and methods. More specifically, the present invention relates to implantable medical leads and methods of manufacturing and implanting such leads.

### BACKGROUND OF THE INVENTION

A recent study has analyzed the impact of left ventricular (“LV”) lead stimulation sites on patient outcomes. The study classified lead position around the perimeter of the LV short axis as anterior, posterior or lateral. With respect to the LV long axis view, the lead positioning was described as apical, mid, or basal.

It could be determined from the study that bi-ventricular (BiV) pacing from a basal lead position is more effective than apical pacing. The study also suggested that posterior lead positioning and lateral lead positioning are better than anterior lead positioning. Accordingly, for the best outcomes, leads should be implanted in the lateral and posterior basal regions.

The lateral and posterial basal locations are regions of last activation. Accordingly, pacing the lateral and posterial basal locations corrects delayed activation and promotes improves resynchronization.

There is a need in the art for implantable medical leads and methods of implantation that facilitate pacing the lateral and posterial basal locations. There is also a need in the art for methods of manufacturing such implantable medical leads.

### BRIEF SUMMARY OF THE INVENTION

Disclosed herein is an implantable medical lead for coupling to an implantable pulse generator and targeted stimulation of the lateral and posterior basal left ventricular region of a patient heart. In one embodiment, the lead includes a lead connector end, a tubular body, at least one electrode and at least one fixation structure. The lead connector end is configured to couple to the implantable pulse generator. The tubular body extends distally from the lead connector end and includes a distal portion distally terminating in a distal end. The at least one electrode is located on the distal portion. The at least one fixation structure is located on the distal portion and includes a bioabsorbable metal. Depending on the embodiment, the bioabsorbable metal includes or is iron, an iron alloy with 35% manganese, or a magnesium alloy. The bioabsorbable metal is configured such that the at least one fixation structure will last long enough at an implantation site so as to secure the distal portion of the tubular body in place via fibrotic tissue.

In one embodiment, the at least one fixation structure is configured to self-bias into an expanded state. The at least one fixation structure may be a single cantilevered loop or multiple cantilevered loops. The cantilevered loop or loops may be formed of a wire formed of the bioabsorbable metal. The at least one fixation structure may be a single bridged loop or multiple bridged loops. The bridged loop or loops may be formed of a wire formed of the bioabsorbable metal.

In one embodiment, the at least one fixation structure may be a stent-like feature. The stent-like feature may be self-expanding or expandable via another mechanism, such as, for example a balloon catheter.

In one embodiment, the at least one fixation structure is located on generally an opposite side of the distal portion of the tubular body from the at least one electrode.

In one embodiment, the distal portion of the tubular body includes a distal section immediately proximal of the distal end and a proximal section extending towards the lead connector end from the distal section. The distal portion of the tubular body includes a curve in the tubular body between the distal section and the proximal section. The at least one fixation structure extends at least substantially along the distal section and the at least one electrode is located on the distal section. In such an embodiment, the fixation structure may extend at least substantially along the distal section on a portion of the tubular body that transitions into an outside of the curve and the electrodes may be located on the distal section on a portion of the tubular body that transitions into an inside of the curve.

While multiple embodiments are disclosed, still other embodiments of the present disclosure will become apparent to those skilled in the art from the following detailed description, which shows and describes illustrative embodiments of the disclosure. As will be realized, the invention is capable of modifications in various aspects, all without departing from the spirit and scope of the present disclosure. Accordingly, the drawings and detailed description are to be regarded as illustrative in nature and not restrictive.

### BRIEF DESCRIPTION OF THE DRAWINGS

FIG. 1 is a diagrammatic depiction of an electrotherapy system electrically coupled to a patient heart as shown in an anterior view, a distal portion of a LV lead being implanted in the CS.

FIGS. 2A and 2B are, respectively, side and top plan views of the distal portion of a first embodiment of a lead tubular body in a free or non-restricted state, the distal portion having a generally straight distal section, a generally straight proximal section proximal of the distal section, and a bioabsorbable cantilevered fixation loop extending distally from a point on the lead tubular body generally between the distal and proximal sections.

FIG. 3 is a side view of the distal portion of a second embodiment of a lead tubular body in a free or non-restricted state, the distal portion having a generally curved distal section, a generally straight proximal section proximal of the distal section, and a bioabsorbable cantilevered fixation loop extending distally from a point on the lead tubular body generally between the distal and proximal sections.

FIGS. 4A and 4B are, respectively, side and top plan views of the distal portion of a third embodiment of a lead tubular body in a free or non-restricted state, the distal portion having a generally straight distal section, a generally straight proximal section proximal of the distal section, and multiple bioabsorbable cantilevered fixation loops positioned on the lead tubular body between the lead tubular body distal end and a point generally between the distal and proximal sections, each cantilevered fixation loop having a single proximal point of contact with the lead tubular body.

FIG. 5 is a side view of the distal portion of a fourth embodiment of a lead tubular body in a free or non-restricted state, the distal portion having a generally curved distal section, a generally straight proximal section proximal of the distal section, and multiple bioabsorbable cantilevered fixation loops positioned on the lead tubular body between the lead tubular body distal end and a point generally between the distal and proximal sections, each cantilevered fixation loop having a single proximal point of contact with the lead tubular body.

FIGS. 6A and 6B are, respectively, side and top plan views of the distal portion of a fifth embodiment of a lead tubular body in a free or non-restricted state, the distal portion having a generally straight distal section, a generally straight proximal section proximal of the distal section, and a bioabsorbable bridging fixation loop extending distally from a point on the lead tubular body generally between the distal and proximal sections to a point on the lead tubular body near or at a distal end of the lead tubular body.

FIG. 7 is a side views of the distal portion of a sixth embodiment of a lead tubular body in a free or non-restricted state, the distal portion having a generally curved distal section, a generally straight proximal section proximal of the distal section, and a bioabsorbable bridging fixation loop extending distally from a point on the lead tubular body generally between the distal and proximal sections to a point on the lead tubular body near or at a distal end of the lead tubular body.

FIGS. 8A and 8B are, respectively, side and top plan views of the distal portion of a seventh embodiment of a lead tubular body in a free or non-restricted state, the distal portion having a generally straight distal section, a generally straight proximal section proximal of the distal section, and multiple bioabsorbable bridging fixation loops positioned on the lead tubular body between the lead tubular body distal end and a point generally between the distal and proximal sections, each bridging fixation loop having a proximal and distal point of contact with the lead tubular body.

FIG. 9 is a side view of the distal portion of a eighth embodiment of a lead tubular body in a free or non-restricted state, the distal portion having a generally curved distal section, a generally straight proximal section proximal of the distal section, and multiple bioabsorbable bridging fixation loops positioned on the lead tubular body between the lead tubular body distal end and a point generally between the distal and proximal sections, each bridging fixation loop having a proximal and distal point of contact with the lead tubular body.

FIGS. 10A and 10B are, respectively, side and top plan views of the distal portion of a ninth embodiment of a lead tubular body in a free or non-restricted state, the distal portion having a generally straight distal section, a generally straight proximal section proximal of the distal section, and an expandable stent positioned on the lead tubular body between the lead tubular body distal end and a point generally between the distal and proximal sections.

FIG. 11 is a side view of the distal portion of a tenth embodiment of a lead tubular body in a free or non-restricted state, the distal portion having a generally curved distal section, a generally straight proximal section proximal of the distal section, and at least one expandable stent positioned on the lead tubular body between the lead tubular body distal end and a point generally between the distal and proximal sections.

FIG. 12 is a left lateral posterior view of the patient heart, the CS extending along the outer surface of the heart between the LV and LA patient left and generally anterior from the OS, anyone of the lead embodiments of FIGS. 2A-11 having its distal portion implanted in the CS and LMV.

FIG. 13 is the same view and lead embodiment as FIG. 12, except the lead distal portion is implanted in the CS and PCV.

FIG. 14 is the same view and lead embodiment as FIG. 12, except the lead distal portion is implanted in the CS and GCV.

### DETAILED DESCRIPTION

Implementations of the present disclosure involve implantable medical leads 5 and methods of implantation that facilitate the targeted stimulation of the lateral and posterior basal region of the heart. The lateral and posterior cardiac veins are good locations for the electrodes 110a-d in order to achieve such targeted stimulation of the lateral and posterior basal region of heart.

Fixation of these leads 5 is achieved by using a fixation structure 115 such as, for example, a flexible wire loop or loops or stent like structure to fix the lead distal portion 50 in the basal region of a coronary vein or one of its associated branches. The fixation structures 115 my be a self-biasing loop, loops, stents or other structures that can be compressed inside a delivery catheter or sheath and then allowed to self-bias into an expanded state when freed from the confines of the delivery catheter or sheath by being pushed out of the delivery catheter or sheath to stabilize the lead in a coronary vein in the basal region of the heart. Alternatively, the fixation structure 115 can be a non-self-biasing structure such as, for example, a stent like structure that is expanded inside the vessel with a balloon supported off of a balloon catheter.

The fixation structures 115 disclosed herein may be formed of a bioabsorbable metal. The use of such materials results in fixation structures 115 of sufficient strength to secure the lead distal end at the implantation site sufficiently long for fibrotic tissue to secure the lead distal end in the vessel before the bioabsorbable metal is fully absorbed into the body. Such bioabsorbable metals are unlikely to embolize, thrombos, or result in extensive inflammatory response. Further, the permanent fixation via fibrotic tissue and the eventual elimination of the metal fixation structure via bioabsorbtion are unlikely to result in occlusion of the vessel.

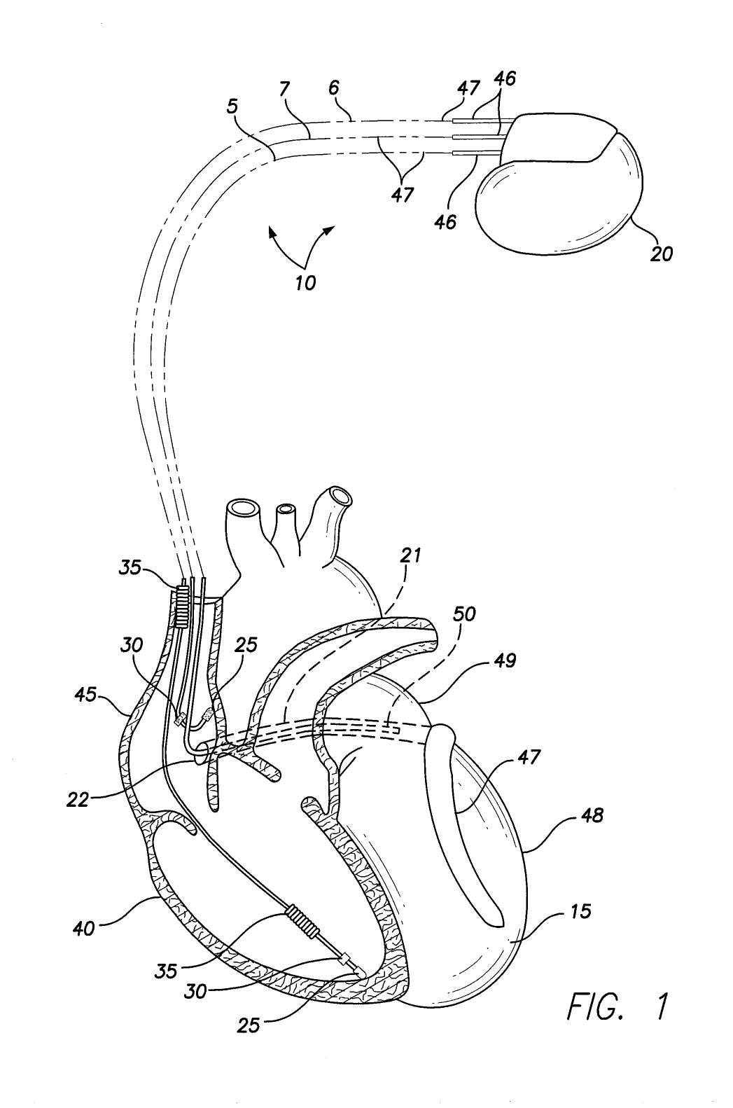

To begin a general, non-limiting discussion regarding some of the features and deployment characteristics common among the various lead and implantation embodiments disclosed herein, reference is made to FIG. 1, which is a diagrammatic depiction of an electrotherapy system 10 electrically coupled to a patient heart 15 as shown in an anterior view. As shown in FIG. 1, the system 10 includes an implantable pulse generator (e.g., pacemaker, implantable cardioverter defibrillator (“ICD”), or etc.) 20 and one or more (e.g., three) implantable medical lead 5, 6, 7 electrically coupling the patient heart 15 to the pulse generator 10. While the following discussion will focus on the configuration and implantation of the left ventricular (“LV”) lead 5 extending into the coronary sinus (“CS”) 21 via the coronary sinus ostium (“OS”) 22, it should be remembered that the system 10 may employ only the LV lead 5 or the LV lead 5 in conjunction with other leads, such as, for example, a right ventricular (“RV”) lead 6 and/or right atrial (“RA”) lead 7. The RV and RA leads 6, 7 may employ pacing electrodes 25, sensing electrodes 30 and shock coils 35 as known in the art to respectively provide electrical stimulation to the right ventricle 40 and right atrium 45 of the heart 15.

Each of the leads 5, 7, 9 is electrically coupled to the pulse generator 20 via a lead connector end 46 at the lead proximal end 47. Electrical conductors (e.g., wires, cables, helically coiled filars, etc.) extend through each lead body from electrical contacts on the lead connector end to the various electrodes and shock coil near the distal region of the lead to electrically couple the various electrodes and shock coil to the pulse generator.

As can be understood from FIG. 1, which shows an anterior view of the patient heart 15, the CS 21 extends generally patient right to patient left from the OS 22 and, further, posterior to anterior until transitioning into the great cardiac vein 47, which then extends in a generally inferior direction along the anterior region of the left ventricle (“LV”) 48. In extending generally posterior to anterior from the OS 22 until transitioning into the great cardiac vein 47, the CS 22 is inferior to the left atrium (“LA”) 49 and superior to the LV 48.

As indicated in FIG. 1, in most embodiments of the LV lead 5 disclosed herein, the distal portion 50 of the LV lead 5 does not extend into the great cardiac vein 47, but is instead implanted in the CS 21 or vein branches extending off of the CS 21, as described in detail below. As explained in the discussion below, the distal portion 50 of each embodiment of the LV lead 5 disclosed herein is configured to facilitate the distal portion 50 being implanted in the CS 21 and, more particularly, in the lateral and posterior basal region of the heart. In other words, most of the embodiments of the LV lead 5 disclosed herein are designed specifically for the stimulation of the basal region of the heart.

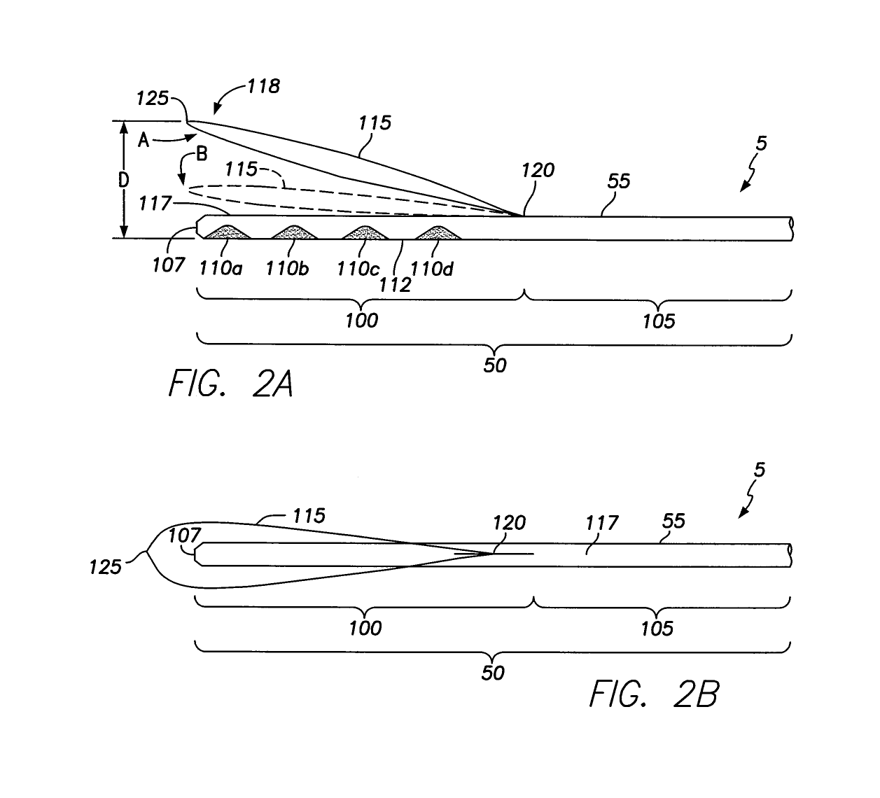

For a discussion of the configuration of the distal portion 50 of a tubular body 55 of a first embodiment of the LV lead 5, reference is made to FIGS. 2A and 2B, which are, respectively, side and top plan views of the distal portion 50 of the lead tubular body 55 in a free or non-restricted state. For purposes of discussion, when the distal portion 50 of the tubular body 55 of the LV lead 5 is said to be in a free or non-restricted state, the distal portion 50 exists in a configuration that the distal portion 50 naturally biases to absent the distal portion 50 being acted upon by an outside force such as, for example, a stylet being extended through the distal portion 50, a sheath or catheter being extended over the distal portion 50, or the distal portion 50 being confined within a vascular structure such as, for example, the CS 21.

As indicated in FIGS. 2A and 2B, the distal portion 50 of the tubular body 55 of the first embodiment of the LV lead 5 includes an extreme distal section 100 and a proximal section 105 that proximally extends from the extreme distal section 100 towards the proximal end 47 of the LV lead 5 that connects to the pulse generator 20 via the lead connector end 46, as can be understood from FIG. 1. The distal section 100 distally terminates as the distal end 107 of the lead 5. The tubular body 55 of the distal section 100 and proximal section 105 are generally linear or straight when in a free or non-restricted state. The tubular body 55 of the distal section 100 and proximal section 105 has a size of between approximately seven French and approximately eight French and is formed of silicone rubber, polyurethane, or silicone rubber polyurethane copolymer (“SPC”).

As shown in FIG. 2A, multiple electrodes 110a-d extend distal-proximal in a spaced-apart manner along a bottom surface 112 of the distal section 100 of the tubular body 55 of the first embodiment of the LV lead 5. These electrodes 110a-d may be employed for pacing, sensing and/or defibrillation purposes. Depending on the embodiment, there may be one, two, three, four or more electrodes 110a-d so located on the bottom surface 112 of the distal section 100 of the lead tubular body. The electrodes 110a-d may extend between approximately two cm and four cm distal to proximal along the lead tubular body. In embodiments of the LV lead 5 where the electrotherapy target for the LV lead 5 is the base of the heart, there is no need for an electrode space that greatly exceeds two cm to four cm. Electrical conductors (e.g., cables, wires, helical coiled filars, etc.) that extend through the tubular body 55 of the LV lead 5 electrically couple the electrodes 100a-d to electrical contacts of the lead connector end 46 of the proximal end 47 of the lead 5 that is received in the pulse generator 20 (see FIG. 1).

As indicated in FIG. 2A, a biasing fixation structure 115 extends from a top surface 117 of the distal section 100 of the lead tubular body. Thus, the biasing fixation structure 115 is generally on an opposite side of the lead tubular body 55 from the electrodes 110a-d. The biasing fixation structure 115 is configured such that at least a portion 118 of the fixation structure 115 biases away from the outer surface of the lead tubular body when the fixation structure 115 in a free or non-restricted state. In other words, at least a portion 118 of the fixation structure 115 naturally assumes or biases into an arrangement wherein the portion 118 is spaced-apart from the outer surface of the lead tubular body 55 absent the fixation structure 115 being acted upon by an outside force such as, for example, a sheath or catheter being extended over the fixation structure 115 and lead tubular body distal portion 50, or the fixation structure 115 and lead tubular body distal portion 50 being confined within a vascular structure such as, for example, the CS 21.

In FIG. 2A, the fixation structure 115 is represented via two different types of lines, namely, a solid line at arrow A and a dashed line at arrow B. The solid line depiction of the fixation structure 115 at arrow A represents the fixation structure 115 when fully biased into its free or non-restricted state. The dashed line depiction of the fixation structure 115 at arrow B represents the fixation structure 115 when confined via, for example, a vascular structure like the CS 21, and thereby prevented from achieving its fully biased state as indicated at arrow A. When fully deployed into its free or non-restricted state as indicated at arrow A, the lead will have a width or diameter D that extends from the bottom surface 112 of the lead tubular body to a point on the fixation structure 115 that is most extremely spaced-apart from the top surface of the lead tubular body. In one embodiment, such a diameter D will be between approximately 9 mm and approximately 18 mm.

As can be understood from FIG. 2A, when the distal section 100 of the lead tubular body distal portion 50 is located within the confines of a vascular structure, such as, for example the CS 21 or one of its branches as discussed below, the walls of the vascular structure confine the fixation structure such that the fixation structure 115 is not able to fully deploy to the state depicted at arrow A. As a result, the fixation structure 115 in attempting to bias into its fully deployed state as indicated by arrow A exerts a force against the wall of the vascular structure, thereby forcing the electrodes 110a-d into excellent electrical contact with cardiac tissue and fixing the distal section 100 of the lead tubular body distal portion 50 in place within the vascular structure.

As illustrated in FIGS. 2A and 2B, the fixation structure 115 has a single point of contact 120 and a free end 125, thereby forming a cantilevered arrangement with the lead tubular body. As shown in FIGS. 2A and 2B, the free end 125 may be distal of contact point 120. In other embodiments, the arrangement may be reversed such that the free end 125 is proximal the contact point 120.

As can be understood from FIG. 3, which is a side view of the distal portion 50 of a lead tubular body 55 in a free or non-restricted state for a second embodiment of the LV lead 5, the lead tubular body 55 may be configured to have a bend or curve as indicated at arrow C. In other words, lead tubular body 55 naturally assumes or biases into a curve at arrow C absent the lead tubular body 55 being acted upon by an outside force such as, for example, a stylet extending through the lead tubular body, a sheath or catheter being extended over the lead tubular body 55, or the lead tubular body 55 being confined within a vascular structure such as, for example, the CS 21. Such a curve as indicated by arrow C may be located at or near the junction between the distal section 100 and the proximal section 105 of the lead tubular body distal portion 50 and extend over the most distal part of the proximal section 105 and the most proximal part of the distal section 100. As can be understood from a comparison of FIGS. 2A and 3, all other aspect of the lead distal portion 50 (e.g., the electrodes, fixation structure, etc.) disclosed therein are substantially the same.

As can be understood from FIG. 3, the fixation structure 115 extends at least substantially along the distal section 100 on a portion of the tubular body 55 that transitions into an outside of the curve indicated by arrow C and the electrodes 115a-d are located on the distal section 100 on a portion of the tubular body 55 that transitions into an inside of the curve indicated by arrow C.

As shown in FIGS. 2A and 2B, the contact point 120 is at or near the transition between the distal section 100 and the proximal section 105. Specifically, the contact point 120 may be just proximal of the most proximal electrode 110d. In addition to being a cantilevered arrangement, the fixation structure 115 may be in the form of a wire loop. The wire forming the loop may have a diameter of between approximately 9 mm and approximately 18 mm and be formed of a bioabsorbable metal such as, for example, iron, iron alloy with 35% manganese, or magnesium alloys.

Although such metal materials are bioabsorbable and will disappear over time subsequent to implantation, fixation structures 115 formed of such bioabsorbable metals will provide adequate biasing force for a period sufficient for the distal section 100 of the lead body distal portion 50 to become secured in place in the vascular structure via tissue growth about the lead distal section 100. Further, such bioabsorbable metals are unlikely to result in embolization, thrombosis, or extensive inflammatory response. Iron alloy with 35% manganese has similar mechanical properties to 316 L stainless steel and has higher in vitro corrosion rates than pure iron. Magnesium alloys do no embolize and are electronegatively charged and, as a result, have minimal thrombogenicity.

Employing bioabsorbable metals for the fixation structures 115 of the leads 5 disclosed herein is advantageous in that the when such leads are implanted in the coronary vein or associated branches, after only a few months the lead is covered with a thin sheath of fibrotic tissue and the coronary vein or associated branches are unlikely to be occluded with blood flowing parallel to the fibrotic tissue sheath covered lead in the lumen of the blood vessel. Once the fibrotic tissue sheath is formed, the lead will be stabilized inside the vein and the fixation structure 115, which is formed of bioabsorbable material, will not be needed as disappears due to be absorbed into the body over time.

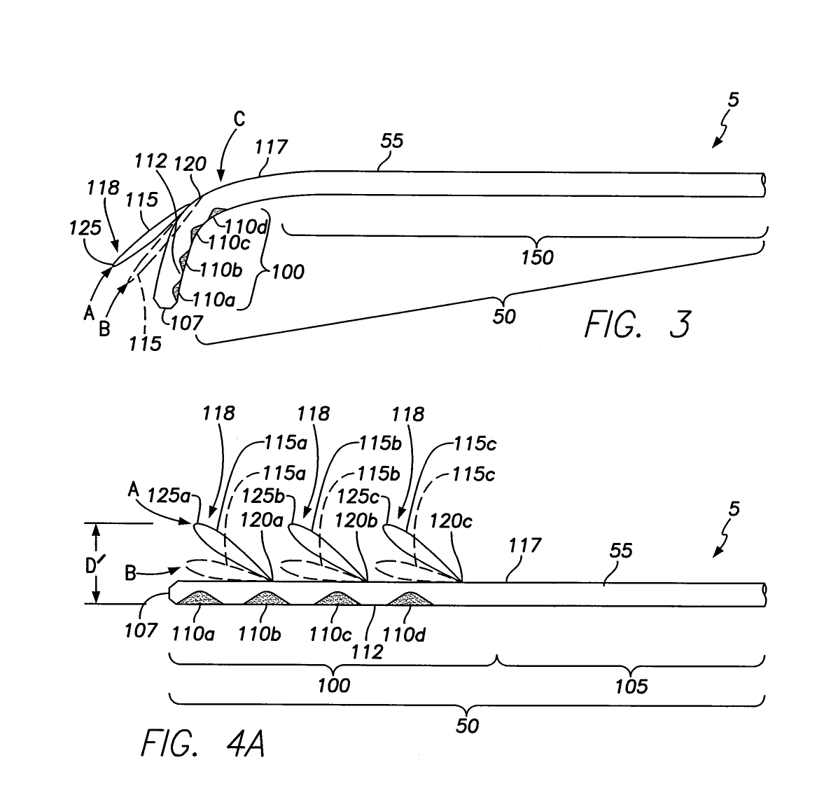

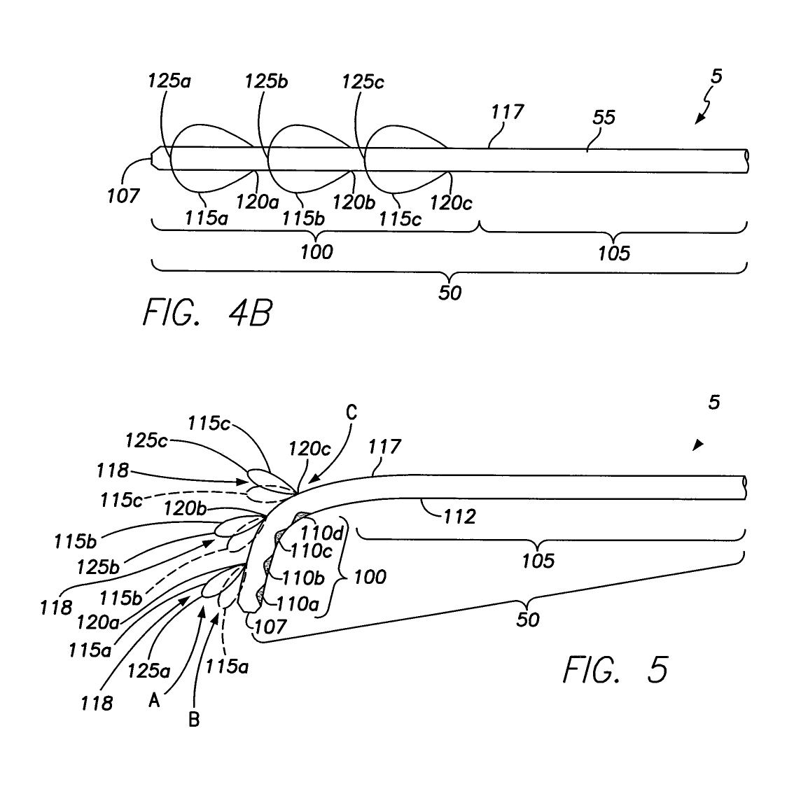

As can be understood from FIGS. 4A and 4B, which are, respectively, side and top plan views of the distal portion 50 of a lead tubular body 55 in a free or non-restricted state for a third embodiment of the LV lead 5, the lead tubular body 55 may have multiple cantilevered fixation structures 115a-c located along the distal section 100 of the lead tubular body distal portion 50 instead of a single cantilevered fixation structure 115 as discussed above with respect to FIGS. 2A and 2B. As can be understood from a comparison of the third embodiment of FIGS. 4A and 4B to the first embodiment of FIGS. 2A and 2B, the electrodes 110a-d of the third embodiment are the same in number and arrangement to the electrodes 110a-d of the first embodiment, and the location of the fixation fixtures 115a-c is also on the opposite side of the lead tubular body 55 from the electrodes 110a-d. There may be two, three, four or more cantilevered fixation structures 115a-c for the third embodiment depicted in FIGS. 4A and 4B.

In FIG. 4A, the fixation structures 115a are represented via two different types of lines, namely, solid lines at arrow A and dashed lines at arrow B. The solid line depiction of the fixation structures 115a-c at arrow A represents the fixation structures 115a-c when fully biased into the free or non-restricted state. The dashed line depiction of the fixation structures 115a-c at arrow B represents the fixation structures 115a-c when confined via, for example, a vascular structure like the CS 21, and thereby prevented from achieving the fully biased state as indicated at arrow A. When fully deployed into the free or non-restricted state as indicated at arrow A, the lead will have a width or diameter D′ that extends from the bottom surface 112 of the lead tubular body to a point 118 on each fixation structure 115a-c that is most extremely spaced-apart from the top surface of the lead tubular body. In one embodiment, such a diameter D′ will be between approximately 5 mm and approximately 18 mm.

As can be understood from FIG. 4A, when the distal section 100 of the lead tubular body distal portion 50 is located within the confines of a vascular structure, such as, for example the CS 21 or one of its branches as discussed below, the walls of the vascular structure confine the fixation structures such that the fixation structures 115a-c are not able to fully deploy to the state depicted at arrow A. As a result, the fixation structures 115a-c in attempting to bias into the fully deployed state as indicated by arrow A exert a force against the wall of the vascular structure, thereby forcing the electrodes 110a-d into excellent electrical contact with cardiac tissue and fixing the distal section 100 of the lead tubular body distal portion 50 in place within the vascular structure.

As illustrated in FIGS. 4A and 4B, each fixation structure 115a-c has a single point of contact 120a-c and a free end 125a-c, thereby forming a cantilevered arrangement with the lead tubular body. As shown in FIGS. 4A and 4B, each free end 125 may be distal of the respective contact point 120a-c. In other embodiments, the arrangement may be reversed such that each free end 125a-c is proximal the respective contact point 120a-c.

As can be understood from FIG. 5, which is a side view of the distal portion 50 of a lead tubular body 55 in a free or non-restricted state for a fourth embodiment of the LV lead 5, the lead tubular body 55 may be configured to have a bend or curve as indicated at arrow C. In other words, lead tubular body 55 naturally assumes or biases into a curve at arrow C absent the lead tubular body 55 being acted upon by an outside force such as, for example, a stylet extending through the lead tubular body, a sheath or catheter being extended over the lead tubular body 55, or the lead tubular body 55 being confined within a vascular structure such as, for example, the CS 21. Such a curve as indicated by arrow C may be located at or near the junction between the distal section 100 and the proximal section 105 of the lead tubular body distal portion 50 and extend over the most distal part of the proximal section 105 and the most proximal part of the distal section 100. As can be understood from a comparison of FIGS. 4A and 5, all other aspect of the lead distal portion 50 (e.g., the electrodes, fixation structures, etc.) disclosed therein are substantially the same.

As shown in FIGS. 4A and 4B, the most proximal fixation structure 115c has a contact point 120c at or near the transition between the distal section 100 and the proximal section 105. Specifically, the contact point 120c of the most proximal fixation structure 115c may be just proximal of the most proximal electrode 110d. The rest of the fixation structures 115a-b may have respective contact points 120a-b that are just distal of the free end 125b-c of the respectively immediately proximal fixation structure 115b-c. In addition to being a cantilevered arrangement, each fixation structure 115a-c may be in the form of a wire loop. The wire forming the loop may have a diameter of between approximately 6 mm and approximately 18 mm and be formed of a bioabsorbable metal such as, for example, iron, iron alloy with 35% manganese, or magnesium alloys.

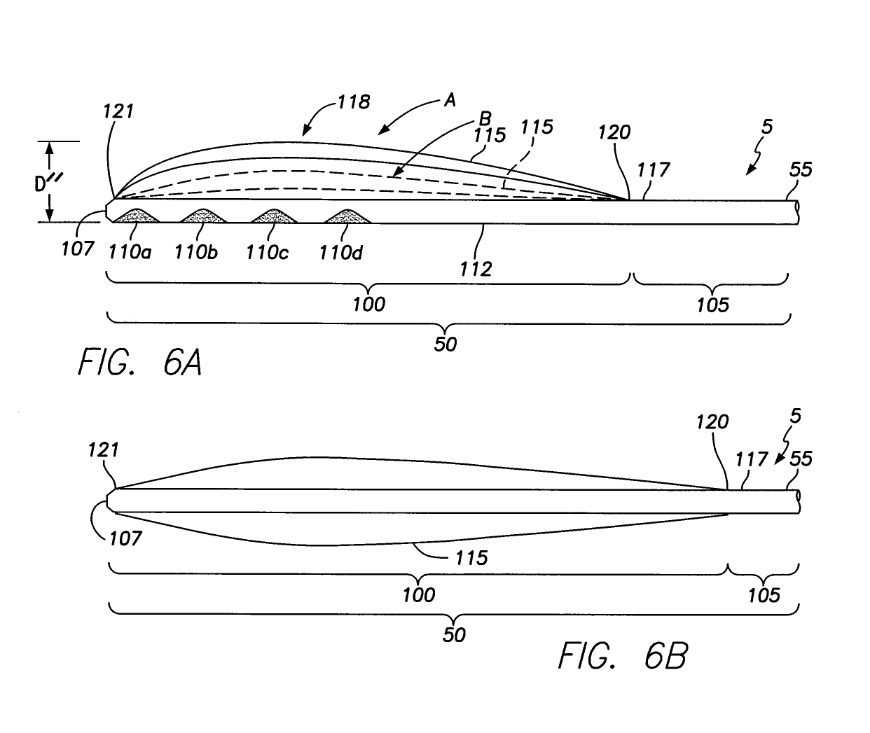

As can be understood from FIGS. 6A and 6B, which are, respectively, side and top plan views of the distal portion 50 of a lead tubular body 55 in a free or non-restricted state for a fifth embodiment of the LV lead 5, the lead tubular body 55 may have a single bridging fixation structure 115 located along the distal section 100 of the lead tubular body distal portion 50 instead of a single cantilevered fixation structure 115 as discussed above with respect to FIGS. 2A and 2B. As can be understood from a comparison of the fifth embodiment of FIGS. 6A and 6B to the first embodiment of FIGS. 2A and 2B, the electrodes 110a-d of the fifth embodiment are the same in number and arrangement to the electrodes 110a-d of the first embodiment, and the location of the fixation fixture 115 is also on the opposite side of the lead tubular body 55 from the electrodes 110a-d.

In FIG. 6A, the fixation structure 115 is represented via two different types of lines, namely, a solid line at arrow A and a dashed line at arrow B. The solid line depiction of the fixation structure 115 at arrow A represents the fixation structure 115 when fully biased into the free or non-restricted state. The dashed line depiction of the fixation structure 115 at arrow B represents the fixation structure 115 when confined via, for example, a vascular structure like the CS 21, and thereby prevented from achieving the fully biased state as indicated at arrow A. When fully deployed into the free or non-restricted state as indicated at arrow A, the lead will have a width or diameter D″ that extends from the bottom surface 112 of the lead tubular body to a point 118 on the fixation structure 115 that is most extremely spaced-apart from the top surface of the lead tubular body. In one embodiment, such a diameter D″ will be between approximately 6 mm and approximately 18 mm.

As can be understood from FIG. 6A, when the distal section 100 of the lead tubular body distal portion 50 is located within the confines of a vascular structure, such as, for example the CS 21 or one of its branches as discussed below, the walls of the vascular structure confine the fixation structure such that the fixation structure 115 is not able to fully deploy to the state depicted at arrow A. As a result, the fixation structure 115 in attempting to bias into the fully deployed state as indicated by arrow A exerts a force against the wall of the vascular structure, thereby forcing the electrodes 110a-d into excellent electrical contact with cardiac tissue and fixing the distal section 100 of the lead tubular body distal portion 50 in place within the vascular structure.

Unlike the cantilevered fixation structure 115 of the embodiment of FIGS. 2A and 2B, the bridging fixation structure 115 of the embodiment of FIGS. 6A and 6B has a fixation structure 115 with a pair of point contacts 120 and 121 as opposed to a single point contact 120 and a free end 125. Since the bridging fixation structure 115 of embodiment of FIGS. 6A and 6B has a proximal point contact 120 and a distal point contact 121 and a free region between these contacts 120 and 121, the result is a fixation structure 115 that is supported off of the lead tubular body 55 at the extreme proximal and distal ends of the fixation structure 115, leaving the extent of the fixation structure 115 to form a bridging type arrangement with the lead tubular body 55.

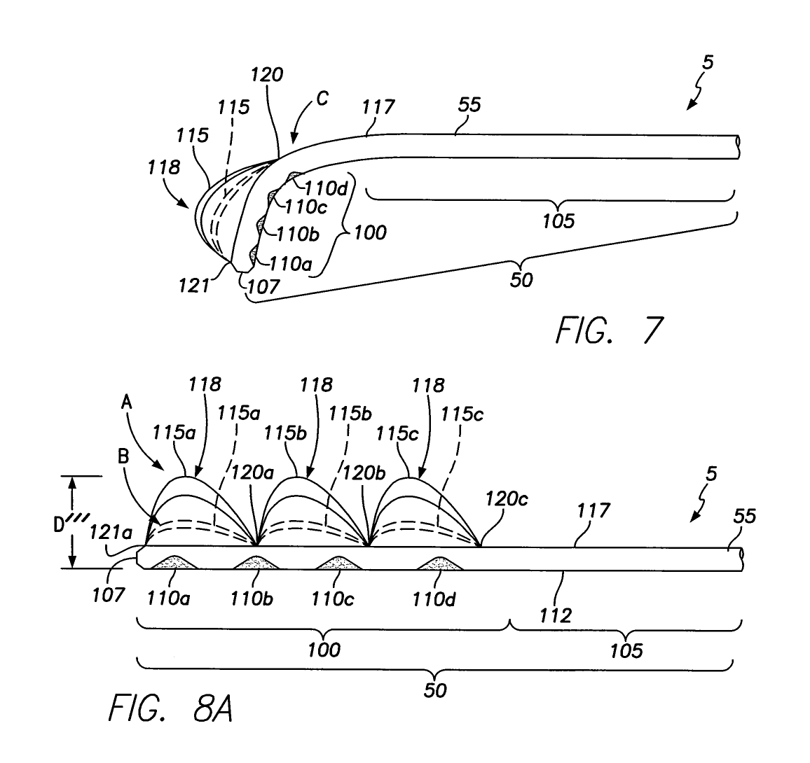

As can be understood from FIG. 7, which is a side view of the distal portion 50 of a lead tubular body 55 in a free or non-restricted state for a sixth embodiment of the LV lead 5, the lead tubular body 55 may be configured to have a bend or curve as indicated at arrow C. In other words, lead tubular body 55 naturally assumes or biases into a curve at arrow C absent the lead tubular body 55 being acted upon by an outside force such as, for example, a stylet extending through the lead tubular body, a sheath or catheter being extended over the lead tubular body 55, or the lead tubular body 55 being confined within a vascular structure such as, for example, the CS 21. Such a curve as indicated by arrow C may be located at or near the junction between the distal section 100 and the proximal section 105 of the lead tubular body distal portion 50 and extend over the most distal part of the proximal section 105 and the most proximal part of the distal section 100. As can be understood from a comparison of FIGS. 6A and 7, all other aspect of the lead distal portion 50 (e.g., the electrodes, fixation structure, etc.) disclosed therein are substantially the same.

As shown in FIGS. 6A and 6B, the bridging fixation structure 115 has a proximal contact point 120 at or near the transition between the distal section 100 and the proximal section 105. Specifically, the proximal contact point 120 of the fixation structure 115 may be just proximal of the most proximal electrode 110d. The bridging fixation structure 115 also has a distal contact point 121 at or near the extreme distal end 107 of the LV lead tubular body 55. Specifically, the distal contact point 121 of the fixation structure 115 may be just distal of the most distal electrode 110a. In addition to being a bridging arrangement, the fixation structure 115 may be in the form of a wire loop. The wire forming the loop may have a diameter of between approximately 6 mm and approximately 19 mm and be formed of a bioabsorbable metal such as, for example, iron, iron alloy with 35% manganese, or magnesium alloys.

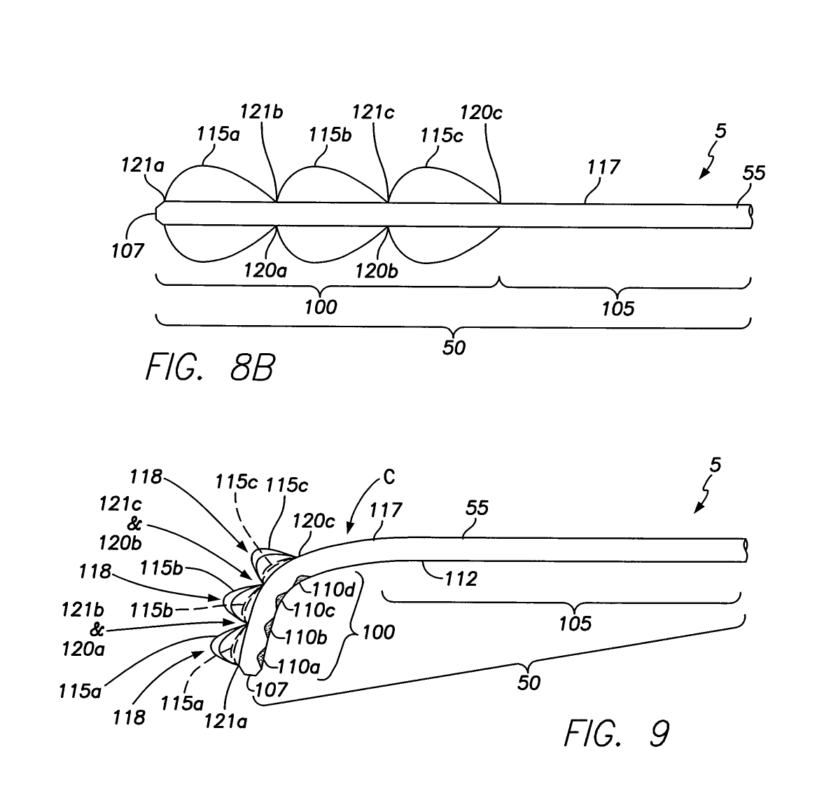

As can be understood from FIGS. 8A and 8B, which are, respectively, side and top plan views of the distal portion 50 of a lead tubular body 55 in a free or non-restricted state for a seventh embodiment of the LV lead 5, the lead tubular body 55 may have multiple bridging fixation structures 115a-c located along the distal section 100 of the lead tubular body distal portion 50 instead of a single bridging fixation structure 115 as discussed above with respect to FIGS. 6A and 6B. As can be understood from a comparison of the seventh embodiment of FIGS. 6A and 6B to the first embodiment of FIGS. 2A and 2B, the electrodes 110a-d of the seventh embodiment are the same in number and arrangement to the electrodes 110a-d of the first embodiment, and the location of the fixation fixtures 115a-c is also on the opposite side of the lead tubular body 55 from the electrodes 110a-d.

In FIG. 8A, the fixation structures 115a-c are represented via two different types of lines, namely, solid lines at arrow A and dashed lines at arrow B. The solid line depiction of the fixation structures 115a-c at arrow A represents the fixation structures 115a-c when fully biased into the free or non-restricted state. The dashed line depiction of the fixation structures 115a-c at arrow B represents the fixation structures 115a-c when confined via, for example, a vascular structure like the CS 21, and thereby prevented from achieving the fully biased state as indicated at arrow A. When fully deployed into the free or non-restricted state as indicated at arrow A, the lead will have a width or diameter D′″ that extends from the bottom surface 112 of the lead tubular body to a point 118 on each fixation structure 115a-c that is most extremely spaced-apart from the top surface of the lead tubular body. In one embodiment, such a diameter D′″ will be between approximately 7 mm and approximately 19 mm.

As can be understood from FIG. 8A, when the distal section 100 of the lead tubular body distal portion 50 is located within the confines of a vascular structure, such as, for example the CS 21 or one of its branches as discussed below, the walls of the vascular structure confine the fixation structures such that the fixation structures 115a-c are not able to fully deploy to the state depicted at arrow A. As a result, the fixation structures 115a-c in attempting to bias into the fully deployed state as indicated by arrow A exert a force against the wall of the vascular structure, thereby forcing the electrodes 110a-d into excellent electrical contact with cardiac tissue and fixing the distal section 100 of the lead tubular body distal portion 50 in place within the vascular structure.

Unlike the multiple cantilevered fixation structures 115 of the embodiment of FIGS. 4A and 4B, each bridging fixation structure 115a-c of the embodiment of FIGS. 8A and 8B has a fixation structure 115a-c with a pair of point contacts 120a-c and 121a-c as opposed to a single point contact 120a-c and a free end 125a-c. Since each bridging fixation fixture 115a-c of the embodiment of FIGS. 8A and 8B has a proximal point contact 120a-c and a distal point contact 121a-c and a free region between these contacts 120 and 121, the result is each bridging fixation structure 115a-c is supported off of the lead tubular body 55 at the extreme proximal and distal ends of the fixation structure 115a-c, leaving the extent of the fixation structure 115a-c to form a bridging type arrangement with the lead tubular body 55.

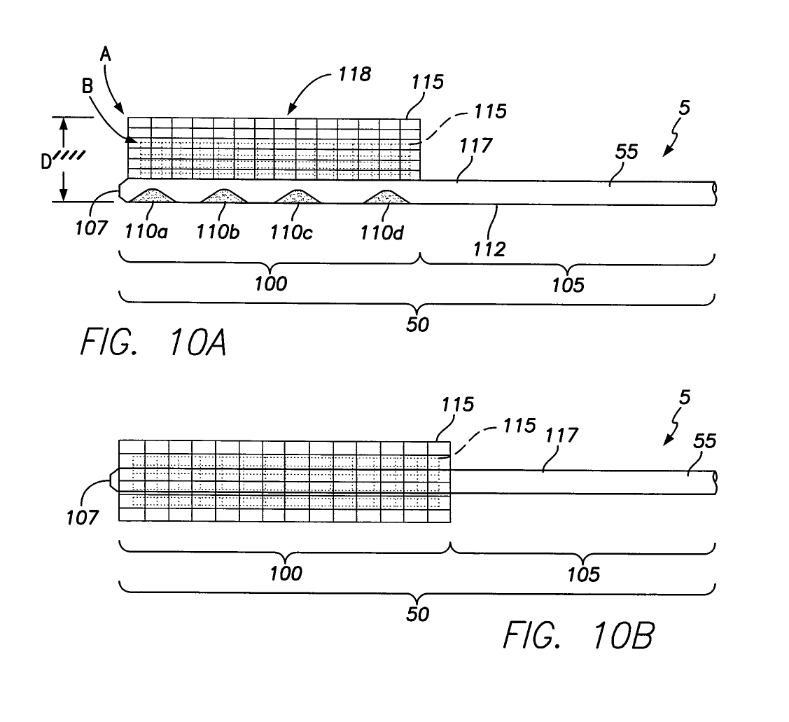

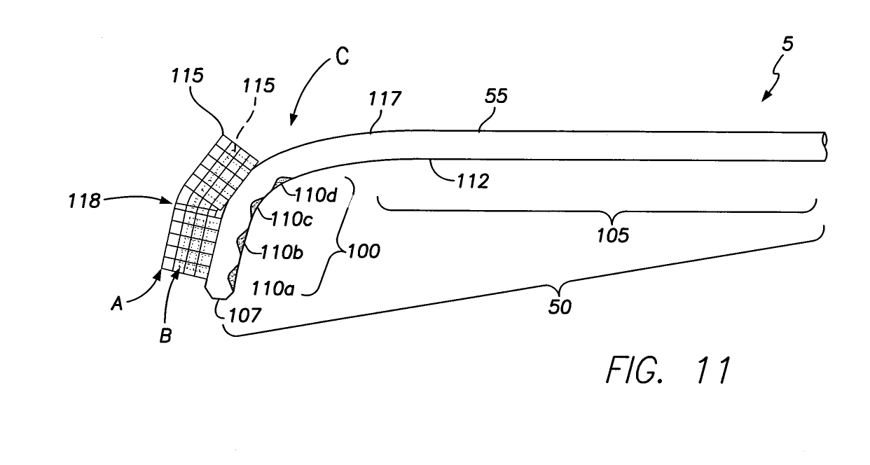

As can be understood from FIG. 9, which is a side view of the distal portion 50 of a lead tubular body 55 in a free or non-restricted state for an eighth embodiment of the LV lead 5, the lead tubular body 55 may be configured to have a bend or curve as indicated at arrow C. In other words, the lead tubular body 55 naturally assumes or biases into a curve at arrow C absent the lead tubular body 55 being acted upon by an outside force such as, for example, a stylet extending through the lead tubular body, a sheath or catheter being extended over the lead tubular body 55, or the lead tubular body 55 being confined within a vascular structure such as, for example, the CS 21. Such a curve as indicated by arrow C may be located at or near the junction between the distal section 100 and the proximal section 105 of the lead tubular body distal portion 50 and extend over the most distal part of the proximal section 105 and the most proximal part of the distal section 100. As can be understood from a comparison of FIGS. 8A and 9, all other aspect of the lead distal portion 50 (e.g., the electrodes, fixation structure, etc.) disclosed therein are substantially the same.

As shown in FIGS. 8A and 8B, the most proximal fixation structure 115c has a proximal contact point 120c at or near the transition between the distal section 100 and the proximal section 105. Specifically, the proximal contact point 120c of the most proximal fixation structure 115c may be just proximal of the most proximal electrode 110d. The most proximal fixation structure 115c also has a distal contact point 121c at or near a proximal contact point 120b of the middle fixation structure 115b. The middle fixation structure 115b also has a distal contact point 121b at or near a proximal contact point 120a of the most distal fixation structure 115a. The most distal fixation structure 115a also has a distal contact point 121a at or near the most distal end 107 of the LV lead tubular body 55. In addition to being a bridging arrangement, each fixation structure 115a-c may be in the form of a wire loop. The wire forming the loop may have a diameter of between approximately 6 mm and approximately 18 mm and be formed of a bioabsorbable metal such as, for example, iron, iron alloy with 35% manganese, or magnesium alloys.

As can be understood from FIGS. 10A and 10B, which are, respectively, side and top plan views of the distal portion 50 of a lead tubular body 55 in a free or non-restricted state for a ninth embodiment of the LV lead 5, the lead tubular body 55 may have a stent-like fixation structures 115 located along the distal section 100 of the lead tubular body distal portion 50. As can be understood from a comparison of the ninth embodiment of FIGS. 10A and 10B to the first embodiment of FIGS. 2A and 2B, the electrodes 110a-d of the ninth embodiment are the same in number and arrangement to the electrodes 110a-d of the first embodiment, and the location of the stent-like fixation fixture 115 is also on the opposite side of the lead tubular body 55 from the electrodes 110a-d.

In FIG. 10A, the stent-like fixation structure 115 is represented via two different types of lines, namely, solid lines at arrow A and dashed lines at arrow B. The solid line depiction of the stent-like fixation structure 115 at arrow A represents the stent-like fixation structure 115 when fully biased into the free or non-restricted state. The dashed line depiction of the stent-like fixation structure 115 at arrow B represents the stent-like fixation structure 115 when confined via, for example, a vascular structure like the CS 21, and thereby prevented from achieving the fully biased state as indicated at arrow A. When fully deployed into the free or non-restricted state as indicated at arrow A, the lead will have a width or diameter D″″ that extends from the bottom surface 112 of the lead tubular body to a point 118 on the sent-like fixation structure 115 that is most extremely spaced-apart from the top surface of the lead tubular body. In one embodiment, such a diameter D″″ will be between approximately 5 mm and approximately 18 mm.

As can be understood from FIG. 10A, when the distal section 100 of the lead tubular body distal portion 50 is located within the confines of a vascular structure, such as, for example the CS 21 or one of its branches as discussed below, the walls of the vascular structure confine the stent-like fixation structure such that the stent-like fixation structure 115 is not able to fully deploy to the state depicted at arrow A. As a result, the stent-like fixation structure 115 in attempting to bias into the fully deployed state as indicated by arrow A exerts a force against the wall of the vascular structure, thereby forcing the electrodes 110a-d into excellent electrical contact with cardiac tissue and fixing the distal section 100 of the lead tubular body distal portion 50 in place within the vascular structure.

In one embodiment, the stent-like fixation structure 115 is coupled to the distal section 100 of the distal portion 50 of the lead tubular body 55 at two or more locations along the stent-like fixation structure. In one embodiment, the stent-like fixation structure 115 has a generally cylindrical configuration. As can be understood from a comparison between the solid lines and the dashed lines respectively illustrating the stent-like fixation structure 115 in expanded and compressed states in FIGS. 10A and 10B, the stent-like fixation structure simply increases in diameter when transitioning from the compressed state to the expanded state. In some embodiments, the stent-like fixation structure 115 of FIGS. 10A-10B is generally self-biasing such that the stent-like structure 115 will bias on its own into the expanded state once freed from the confines a restricting structure (e.g., stylet, catheter, sheath, vessel wall, etc.).

In other embodiments, the stent-like fixation structure 115 of FIGS. 10A-10B is expanded by application of an expanding force provided via a separate device such as, for example, the application of an expanding balloon on a balloon catheter used to deliver the lead distal end 107 to the implantation site. Once the stent-like fixation structure 115 is expanded via the balloon, the stent-like balloon structure maintains on its own the expanded diameter, and the balloon and supporting catheter can be withdrawn from the permanently expanded stent-like fixation structure 115.

Regardless of whether the stent-like fixation structure 115 is self-expanding or balloon-expanded, in some embodiments, the stent-like fixation structure 115 is formed of a bioabsorbable metal such as, for example, iron, iron alloy with 35% manganese, or magnesium alloys.

As can be understood from FIG. 11, which is a side view of the distal portion 50 of a lead tubular body 55 in a free or non-restricted state for an tenth embodiment of the LV lead 5, the lead tubular body 55 may be configured to have a bend or curve as indicated at arrow C. In other words, the lead tubular body 55 naturally assumes or biases into a curve at arrow C absent the lead tubular body 55 being acted upon by an outside force such as, for example, a stylet extending through the lead tubular body, a sheath or catheter being extended over the lead tubular body 55, or the lead tubular body 55 being confined within a vascular structure such as, for example, the CS 21. Such a curve as indicated by arrow C may be located at or near the junction between the distal section 100 and the proximal section 105 of the lead tubular body distal portion 50 and extend over the most distal part of the proximal section 105 and the most proximal part of the distal section 100. As can be understood from a comparison of FIGS. 10A and 11, all other aspect of the lead distal portion 50 (e.g., the electrodes, fixation structure(s), etc.) disclosed therein are substantially the same.

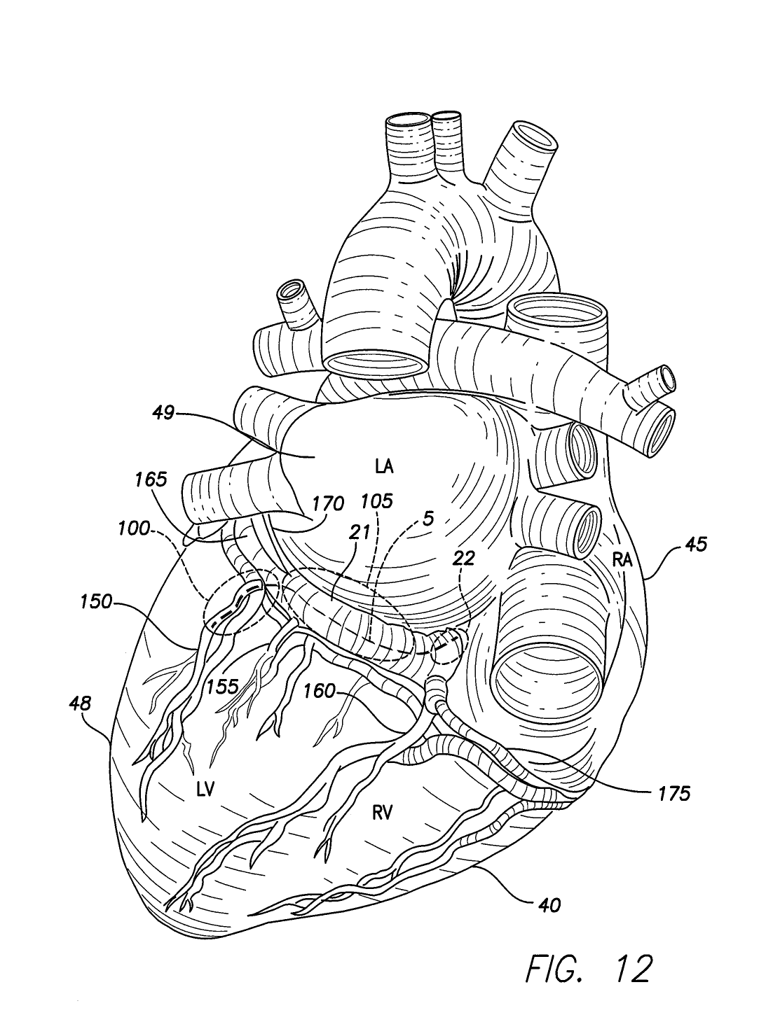

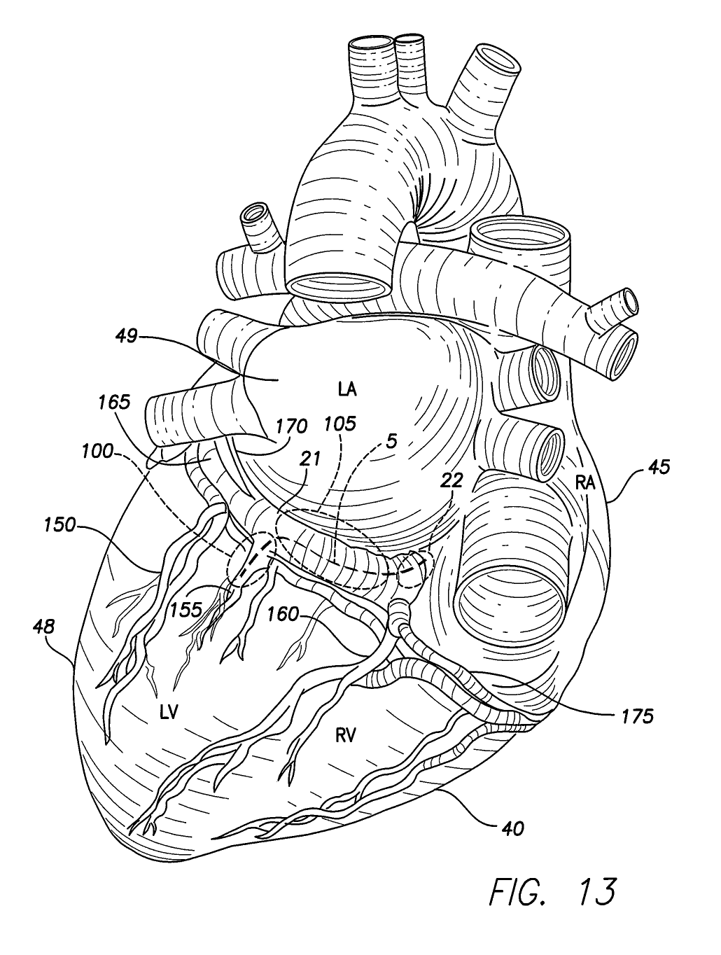

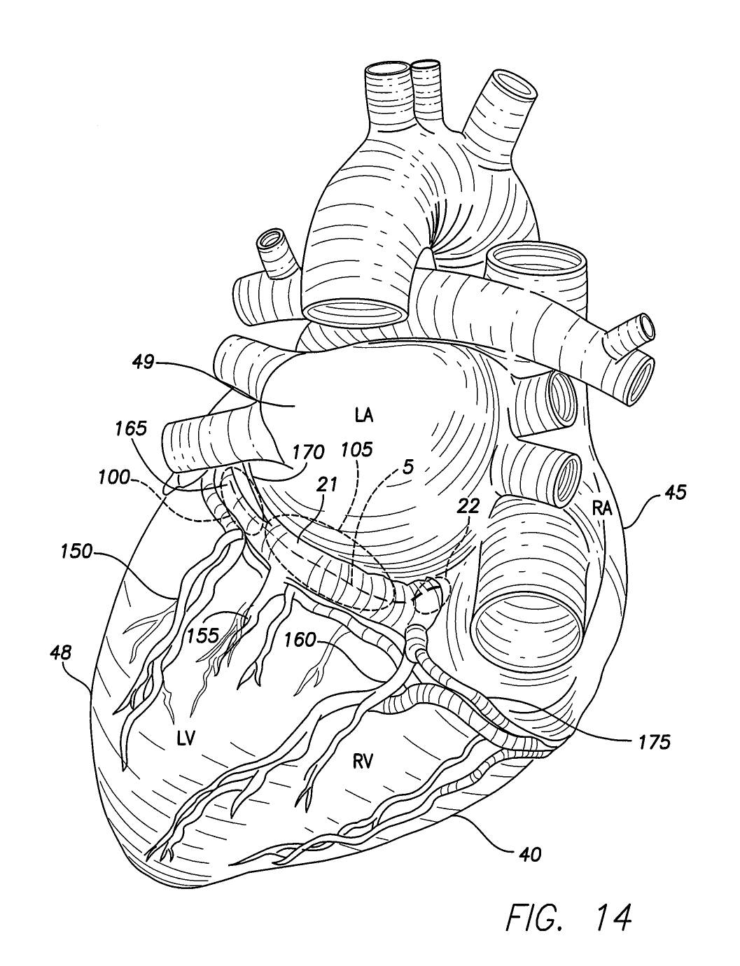

As shown in FIGS. 12-14, which are each left lateral posterior views of the patient heart 15, the CS 21 extends along the outer surface of the heart 15 between the LV 48 and LA 49 patient left and generally anterior from the OS 22. A left marginal cardiac vein (“LMV”) 150, a posterior cardiac vein (“PCV”) 155 and a middle cardiac vein (“MCV”) 160 extend generally inferior off of the CS 21. A great cardiac vein (“GCV”) 165 can be seen to extend generally anterior from the CS 21, and a vein of Marshall (“VM”) 170 can be seen to extend generally anterior from the CS 21 and superior the GCV 165. A small cardiac vein (“SCV”) 175 can be seen to extend patient right and generally posterior off of the CS 21. Depending on the individual, the mid-coronary sinus, which extends from the MCV 160 to the PCV 155, may have a diameter between approximately eight millimeters and approximately ten millimeters. Depending on the individual, the distal-coronary sinus, which extends from the PCV 155 to the GCV 165, may have a diameter between approximately five millimeters and approximately seven millimeters.

As can be understood from FIGS. 12-14, in various embodiments, the LV lead 5 can be delivered into the CS 21 and the LMV 150, the PCV 155 or the GCV 165 via one or more delivery tools (e.g., stylets, guidewires, sheaths, catheters, etc.). When the delivery tools are removed from the distal portion 50 (see FIGS. 2A-11), each fixation structure 115 (115a-c) of the distal section 100 of the distal portion 50 of the LV lead 5 is free to bias against the inner wall surface of the LMV 150, PCV 155 or GCV 165, as the case may be. As a result, each fixation structure 115 (or combination of fixation structures 115a-c) exerts a force F of between approximately 8,000 dynes to approximately 36,000 dynes for each centimeter the fixation structure 115 (or combination of fixation structures 115a-c) is compressed by the inner wall surface of the vessel (i.e., LMV 150, PCV 155 or GCV 165) from the free or non-restricted state distance D, D′, D″, D′″ or D″″ as the case may be with respect to the fixation structure 115 (or combination of fixation structures 115a-c) being any one of the embodiments discussed above with respect to FIGS. 2A-11. The distal section 100 extends into the LMV 150, PCV 155 or GCV 165, as the case may be, in a generally linear arrangement with the electrodes 110a-d oriented so as to generally face the cardiac tissue.

While the bioabsorbable fixation structures 115 (115a-c) will eventually absorb into the patient so as to disappear from the above-discussed implantation sites in the LMV 150, PCV 155 and GCV 165, the bioabsorbable fixation structures will last long enough at the implantation sites so as to secure the distal section 100 of the LV lead tubular body 55 in place via fibrotic tissue that will incase the lead distal section 100.

The foregoing merely illustrates the principles of the invention. Various modifications and alterations to the described embodiments will be apparent to those skilled in the art in view of the teachings herein. It will thus be appreciated that those skilled in the art will be able to devise numerous systems, arrangements and methods which, although not explicitly shown or described herein, embody the principles of the invention and are thus within the spirit and scope of the present invention. From the above description and drawings, it will be understood by those of ordinary skill in the art that the particular embodiments shown and described are for purposes of illustrations only and are not intended to limit the scope of the present invention. References to details of particular embodiments are not intended to limit the scope of the invention.

What is claimed is:

1. An implantable medical lead for coupling to an implantable pulse generator and targeted stimulation of the lateral and posterior basal left ventricular region of a patient heart, the lead comprising:
a lead connector end configured to couple to the implantable pulse generator;
a tubular body extending distally from the lead connector end and comprising a distal portion distally terminating in a distal end;
at least one electrode located on the distal portion; and
at least one fixation structure located on the distal portion and comprising a bioabsorbable metal.

2. The lead of claim 1, wherein the bioabsorbable metal comprises iron.

3. The lead of claim 1, wherein the bioabsorbable metal comprises an iron alloy with 35% manganese.

4. The lead of claim 1, wherein the bioabsorbable metal comprises a magnesium alloy.

5. The lead of claim 1, wherein the bioabsorbable metal is configured such that the at least one fixation structure will last long enough at an implantation site so as to secure the distal portion of the tubular body in place via fibrotic tissue.

6. The lead of claim 1, wherein the at least one fixation structure is configured to self-bias into an expanded state.

7. The lead of claim 6, wherein the at least one fixation structure comprises a cantilevered loop.

8. The lead of claim 7, wherein the cantilevered loop comprises a wire comprising the bioabsorbable metal.

9. The lead of claim 6, wherein the at least one fixation structure comprises multiple cantilevered loops.

10. The lead of claim 6, wherein the at least one fixation structure comprises a bridged loop.

11. The lead of claim 10, wherein the bridged loop comprises a wire comprising the bioabsorbable metal.

12. The lead of claim 6, wherein the at least one fixation structure comprises multiple bridged loops.

13. The lead of claim 1, wherein the at least one fixation structure comprises a stent-like feature.

14. The lead of claim 12, wherein the stent-like feature is self-expanding.

15. The lead of claim 12, wherein the stent-like feature is configured to be expanded via a balloon.

16. The lead of claim 1, wherein the at least one fixation structure is located on generally an opposite side of the distal portion of the tubular body from the at least one electrode.

18. The lead of claim 1, wherein the at least one electrode comprises four electrodes.

19. The lead of claim 1, wherein the distal portion of the tubular body includes a distal section immediately proximal of the distal end and a proximal section extending towards the lead connector end from the distal section, wherein the at least one fixation structure extends at least substantially along the distal section and the at least one electrode is located on the distal section.

20. The lead of claim 19, wherein the distal portion of the tubular body includes a curve in the tubular body between the distal section and the proximal section and the at least one fixation structure extends at least substantially along the distal section on a portion of the tubular body that transitions into an outside of the curve and the at least one electrode is located on the distal section on a portion of the tubular body that transitions into an inside of the curve.
